(19) United States
(12) Patent Application Publication
(10) Pub. No.: US 2013/0231555 A1
(43) Pub. Date: Sep. 5, 2013

(54) IMAGE GUIDED SURGERY APPARATUS AND SYSTEM

(71) Applicants: Shinn-Zong LIN, Taichung City (TW); Jin-Chern CHIOU, Hsinchu City (TW); Horng-Jyh HARN, New Taipei City (TW); Jeng-Ren DUANN, Taoyuan County (TW); Yung-Jiun LIN, Miaoli County (TW)

(72) Inventors: Shinn-Zong LIN, Taichung City (TW); Jin-Chern CHIOU, Hsinchu City (TW); Horng-Jyh HARN, New Taipei City (TW); Jeng-Ren DUANN, Taoyuan County (TW); Yung-Jiun LIN, Miaoli County (TW)

(73) Assignee: CHINA MEDICAL UNIVERSITY, Taichung City (TW)

(21) Appl. No.: 13/615,610

(22) Filed: Sep. 14, 2012

(30) Foreign Application Priority Data
Mar. 3, 2012 (TW) 101107199

(51) Int. Cl. A61M 25/095

(52) U.S. Cl. 600/424

(57) ABSTRACT

An image guided surgery apparatus and system are provided. The image guided surgery system includes an image capturing system, an image scanner, a navigator, a surgical catheter and a surgical probe. The image capturing system has an elongated and flexible working tube for capturing a surface image, a structure image or a dynamic structure image of a tissue during the surgical catheter moving forward to the surgical part, thus determining the type and the structure of the tissue in the front-end of the surgical catheter. Therefore, whether a moving path of the surgical catheter is to at the correct position can be confirmed for reaching a correct surgical site. The surgical catheter assists the surgical probe directly reaching the optimal surgical site for performing a precise positioning surgery.

### RELATED APPLICATIONS

The application claims priority to Taiwan Application Serial Number 101107199, filed Mar. 3, 2012, which is herein incorporated by reference.

### BACKGROUND

1. Field of Invention

The present invention relates to a probe apparatus and a system including the probe apparatus. More particularly, the present invention relates to an image guided surgery apparatus combing an image scanning technology with a navigation technology and a system including the same.

2. Description of Related Art

Nowadays, common human brain diseases include brain tumors, Parkinson's disease, epilepsy, etc. These diseases often easily cause a patient to involuntarily tremble, have a headache, vomit, have visual disturbance, have confusion, have body movement disability and other symptoms, thus significantly reducing the patient's life function and quality, even directly endangering the patient's life. However, when this type of patient undergoes a conservative treatment such as medication or rehabilitation, etc., and is still difficult to recover, an invasive surgery is commonly used as an ultimate treatment. A doctor has to select a very small surgical site from a patient's brain nerve, and use a surgical probe to perform a thermal ablation treatment, such as a surgical apparatus disclosed in Taiwan Patent Publication Number 200526171 entitiled “APPARATUS FOR THE TREATMENT OF HOLLOW ANATOMICAL STRUCTURES”.

FIG. 1 is a schematic view showing a conventional probe surgery system. Based on the conventional invasive brain surgery, a MRI (Magnetic Resonance Imaging) image 700 is generated by nuclear magnetic resonance before surgery, and the MRI image 700 is used for establishing a virtual path plan used for positioning a positioning frame 701, and a surgical probe 702 collaborated with the positioning frame 701 is moved close to a surgical site. At this point, the surgical probe 702 returns 3D positioning signals (three-dimensional positioned location) continuously to a 3D positioning device 703, and the information of the 3D positioned location is transmitted to a master computer of the 3D positioning device 703, thus estimating the surgical site of the patient's brain and counterpointing the surgical site of the patient's brain and the CT (Computed Tomography) MRI image 700, and when the counterpointing is completed, the doctor may perform a subsequent computer-aided guided surgery. This 3D positioning may provide the doctor with the determination of the surgical site and angle, and the surgical probe 702 may be slightly adjusted in accordance with the doctor's experience.

For example, one of the conventional technology of a positioning probe includes a main body with a triangular shape and a sensor placed on each vertex of the main body, and a centroid of the main body is used for computing a three-dimensional axle center, thus generating a virtual three-dimensional space. In addition, a plurality of image positioning camera lens are installed, and a plurality of positioning measuring points are set on the probe, and thus three-dimensional data of the probe obtained from the image positioning camera lens and the virtual three-dimensional space are used to perform navigation and computation. Although the probe positioning and navigation method disclosed here may perform navigation and positioning, yet when the doctor perform an access point operation, since different people have different shapes and sizes of brains, errors are often generated due to the difference between of the virtual three-dimensional space and the surgical site. In addition, the three-dimensional positioning signal of the probe also is the possible surgical site obtained by computation.

For another example, a light ball positioning probe of branch type developed by Medtronic Company (Minnesota, USA) is to use a space area surrounded by 5 light balls to generate a virtual three-dimensional space, which also computes the three-dimensional position, and a computation error aslo still exists in the position confirmation operation. In addition, the light balls are the access points of passive type signals, and thus can be interfered by user differences or a sheltering effect of surrounding environment.

Taiwan Publication Number 200833293 entitled “WIRE ESS POSITIONING PROBE WITH CONTINUOUS ACCESS POINT AND THE POSITIONING METHOD OF THE SAME” is presented to improve the complicated operation steps. This probe provides a probe connector with a quick release feature for fixing or releasing different types of probes, and thus the doctor may use different types of probes in accordance with different surgical needs, and also the released probes can be autoclaved for reducing the risk of infection, in addition, the probe connector can be easily dismantled and assembled without needing to use additional tools, and also can perform an angle alignment for use convenience. The positioning probe provides a functional component containing a compressed continuous access point used for selecting a continuous characteristic (this characteristic refers to the three-dimensional signals or neural interface echo) such that the operation of taking access points by using a push-button remote control can he performed without a doctor or assistant's assistance. Such an active sensing wireless transmitter is used for allowing the doctor to operate the probe conveniently in an operated space for transmitting the three-dimensional data.

Based on the above description, if the probe position is computed by the three-dimensional space, the surgical site is still an estimating position which is obtained indirectly, and thus the accuracy is indeed not easy to be improved. The mode of using the positioning probe to select continuous characteristic (the characteristic refers to the three-dimensional positioning or neural interface echo) is also to estimate the surgical site by computation. In addition, even if the probe can be easily dismantled and assembled without needing to use additional tools, the precise positioning is not benefited. Therefore, since the design of the conventional guided probe does not help much for performing surgery, the current surgery operated within a human body is still like operating the surgery blindly, and thus the problem that the computed three-dimensional data cannot fully meet the requirements of surgery operation still exists.

### SUMMARY

Therefore, the present invention provides an image guided surgery apparatus. The image guided surgery apparatus has an image capturing system for capturing a surface image, a structure image or a dynamic structure image of a tissue, and the image capturing system has an elongated and flexible working tube, wherein when the image capturing system is collaborated with a surgical catheter and a surgical probe, the working tube of the image capturing system will assist the surgical catheter to reach a correct surgical site, and the surgical catheter will assist the surgical probe to directly reach the optimal surgical site for surgery.

According to an embodiment of the present invention, an image guided surgery apparatus includes a positioning frame, a surgical catheter, an image capturing system and a surgical probe. The surgical catheter has a hollow guide with two open ends and controllable curvature, wherein the surgical catheter is disposed and positioned on the positioning frame. The image capturing system has a working tube and a main body which are connected, wherein the working tube shows a probe shape and is flexible for obtaining a real-time image, and the working tube is stretchable freely in the hollow guide of the surgical catheter. The surgical probe has a surgical end and a needle body which are connected and stretchable freely in the hollow guide of the surgical catheter for directly reaching an optimal surgical site for surgery.

In the embodiment, the surgical end of the surgical probe has a thermal ablation function for removing of a surgical part; a measurement function for physiologically measuring the surgical part; a stimulation function for physiologically stimulating the surgical part; a release or clench function for releasing or clenching an implant; or a combination of the aforementioned functions.

In addition, in the embodiment, the working tube of the capturing system is an image capturing unit, and the image unit includes an excitation source and a receiver, thereby capturing a surface image, a structure image or a dynamic structure image of the tissue of the surgical part, thus generating an actual image of the surgical part.

The present invention provides a probe image guided surgery system, The probe image guided surgery system has an image scanner, a navigator, a surgical catheter, an image capturing system and a surgical probe, thereby performing a precise positioning surgery.

According to an embodiment of the present invention, a probe image guided surgery system is collaborated with a positioning frame, and the probe image guided surgery system includes an image scanner, a surgical catheter, an image capturing system, a surgical probe and a navigator. The image scanner is used for obtaining at least one base image of a surgical part and generating a path plan. The surgical catheter has a hollow guide with two open ends and controllable curvature, wherein the surgical catheter is disposed and positioned on the positioning frame, and one end of the surgical catheter reaches around the surgical part in accordance with the path plan. The image capturing system has a working tube and a main body which are connected, wherein the working tube is stretchable freely in the hollow guide of the surgical catheter, and the working tube is used for capturing a surface image, a structure image or a dynamic structure image of the front-end tissue. The surgical probe has a surgical end and a needle body which are connected and stretchable freely in the hollow guide of the surgical catheter. The navigator is connected to the image capturing system and displays a real-time image. Through the image capturing system, the surgical catheter can move forward to the optimal surgical site in accordance with the path plan of the image scanner, and when the surgical catheter is fixed, the surgical probe passes through the surgical catheter to reach the optimal surgical site, and thereafter the navigator is collaborated with the surgical probe for performing a precise positioning surgery.

Based on the above description, it is worthy to be noted that, a non-invasive tissue imaging device using magnetic resonance(MR), X-ray, CT scan, supersonic wave, etc. can be applied as the image scanner for obtaining a tissue image provided for subsequent path planning. In addition, the aforementioned image capturing system has an excitation source and a receiver. The excitation source is used to emit visible light, invisible light, electromagnetic wave or supersonic wave, and the receiver is used to convert a signal of visible light, invisible light, electromagnetic wave or supersonic wave through reflection or diffraction of the tissue at the surgical part to form a real-time image.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a schematic view of a conventional probe surgery system;

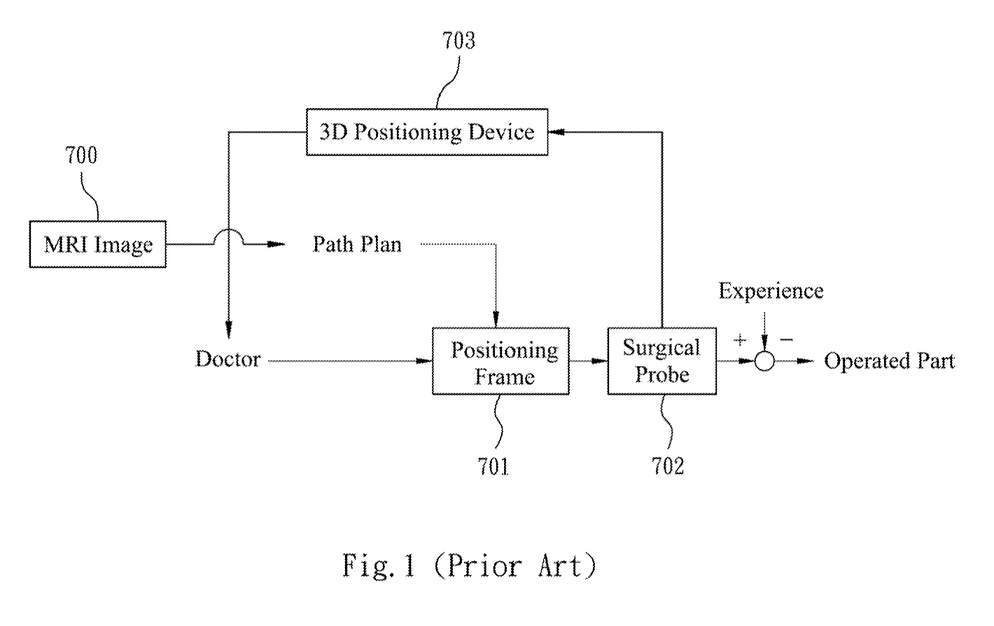

FIG. 2 is a schematic view according to one embodiment of the present invention;

FIG. 3 is a schematic partial exploded three-dimensional view according to one embodiment of the present invention; and

FIG. 4 is a schematic view showing an operating state of the present invention.

### DETAILED DESCRIPTION

Reference will now be made in detail to the present embodiments of the present invention, examples of which are illustrated in the accompanying drawings. Wherever possible, the same reference numbers are used in the drawings and the description to refer to the same or like parts.

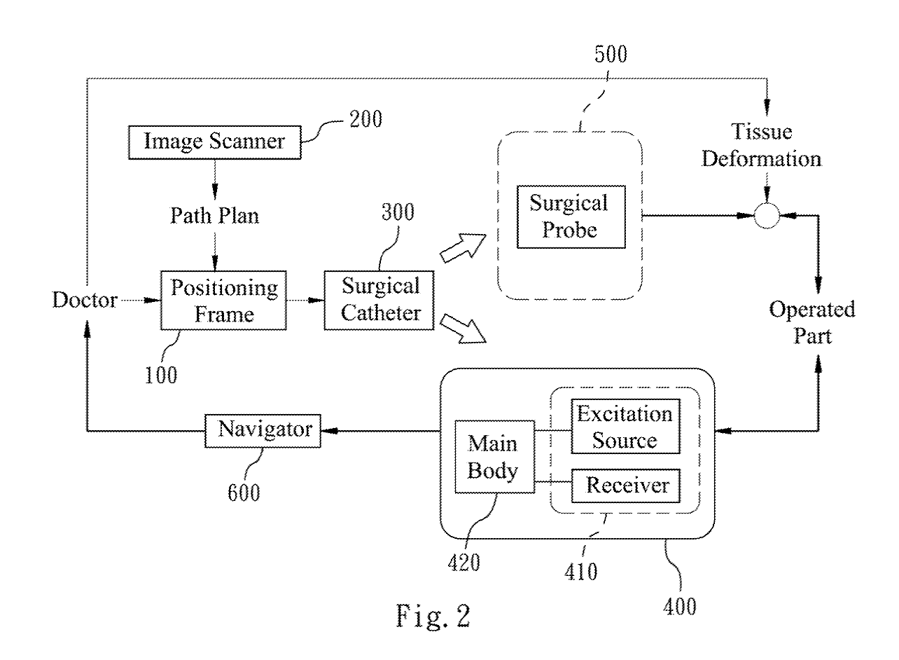

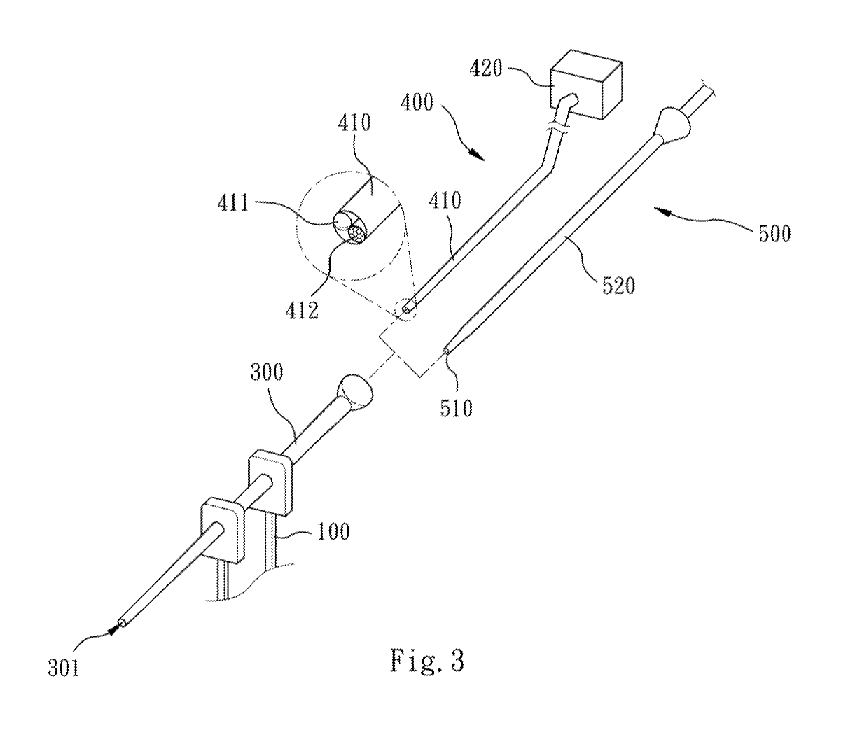

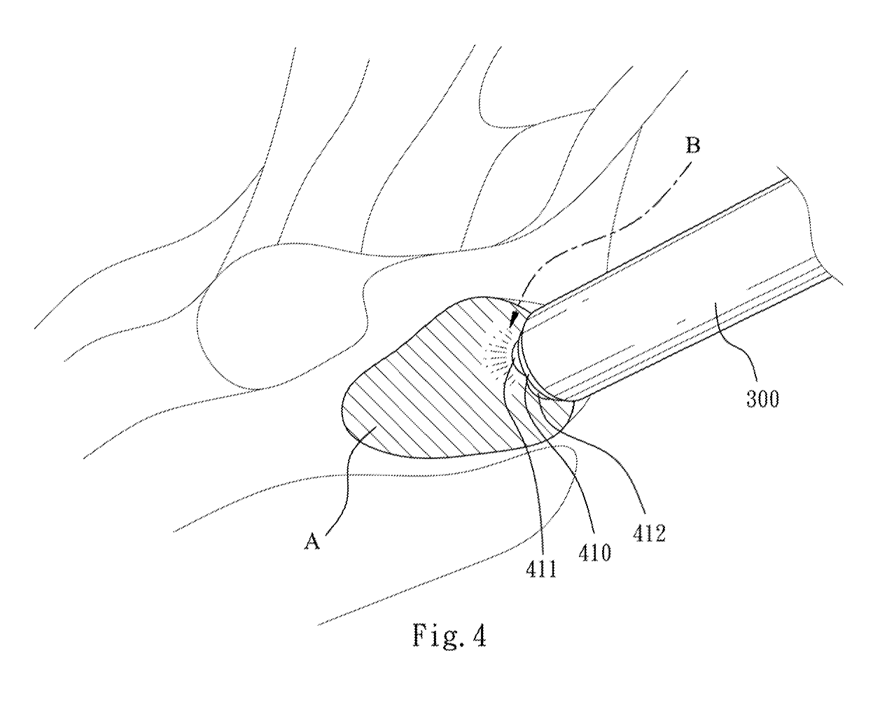

Referring to FIG. 2, FIG. 2 illustrates a schematic view according to one embodiment of the present invention. In addition, FIG. 3 is a schematic partial exploded three-dimensional view according to one embodiment of the present invention, and FIG. 4 is a schematic view showing an operating state of the present invention.

According to an embodiment of the present invention, in a common invasive surgery of Parkinson's disease, a doctor has to place an electrode plate on subthalamic nucleus and performs electrical stimulation onto the brain's deep structure of a patient, or performs a thermal ablation to remove globus pallidus or thalamus. An image guided surgery apparatus and a system with the image guided surgery apparatus include the following components.

A positioning frame 100 is disposed on a predetermined location.

An image scanner 200 is used for obtaining at least one base image for example, Magnetic Resonance Imaging (MRI)) before surgery, and the base image is used to generate a virtual path plan for moving the image scanner 200 forward to a surgical part A, and the virtual path plan is used for allowing the doctor to confirm the predetermined location and the angle of the positioning frame 100.

A surgical catheter 300 has a hollow guide 301 with two open ends and controllable curvature, and the surgical catheter 300 is disposed and positioned on the positioning frame 100, and one end of the surgical catheter 300 reaches around the surgical part A in accordance with the path plan of the image scanner 200.

An image capturing system 400 has a working tube 410 and a main body 420 which are connected, wherein the working tube 410 shows a probe shape and is flexible, and the working tube 410 is stretchable freely in the hollow guide 301 of the surgical catheter 300. The working tube 410 has an image capturing unit composed of an excitation source 411 and a receiver 412, wherein the receiver 412 is used to receive a return signal of reflection or diffraction which is emitted to the surgical part A by the excitation source 410, and the return signal is transmitted back to main body 420, thus generating a surface image, a structure image or a dynamic structure image of the tissue of the actual surgical part A. The excitation source 410 is used to emit an excitation signal B to the surgical part A, wherein the excitation signal B can be a signal of light wave, supersonic wave, electromagnetic wave or other energies, and the return signal can be a signal of light wave, supersonic wave, electromagnetic wave, thermal radiation or plural energies. The excitation signal B emitted by the excitation source 411 and the return signal received by the receiver 412 can be different types. For example, if the excitation source 411 emits light wave, the receiver 412 may receive supersonic wave. If the signal emitted by the excitation source 411 and the return signal received by the receiver 412 both are light waves, the excitation source 411 and the receiver 412 may be the same optical fiber since light can be transmitted within the same optical path without mutual interference. The excitation source 411 and the receiver 412 may further include a scanning device used for performing a scanning in a forward direction or a lateral direction to the working tube 410, thus generating the structure image or the dynamic structure image.

A surgical probe 500 has a surgical end 510 and a needle body 520 which are connected. The surgical end 510 has functions of thermal ablation, measurement, stimulation, release or clench for surgery, wherein the surgical end 510 can perform one single function or a plurality of the functions, and the needle body 520 is stretchable freely in the hollow guide 301 of the surgical catheter 300.

A navigator 600 is connected to the image capturing system 400 for obtaining a real-time image (a surface image, a structure image or a dynamic structure image of the tissue), and the navigator 600 has a display (not shown in Fig) for simultaneously or overlappingly displaying the real-time image with the base image and the path plan generated by the image scanner 200. Accordingly, the real-time image can assist the doctor to determine a surgical site for the surgical probe 500 to reach, and the doctor can perform a precise positioning surgery.

By using the image guided surgery apparatus of the present invention, in operation, the surgical catheter 300 is disposed and positioned on the positioning frame 100, and is collaborated with MRI (Magnetic Resonance Imaging) generated by image scanner 200, and thus the front-end of the surgical catheter 300 may be moved forward to the surgical part A in accordance with the virtual path plan. While being moved forward to the surgical part A. the surgical catheter 300 stops moving at each checkpoint of the virtual path plan, and the working tube 410 of the image capturing system 400 is stretchable freely in the hollow guide 301 of the surgical catheter 300 for capturing a surface image, a structure image or a dynamic structure image of the tissue at each checkpoint, thereby confirming the accuracy of the path plan. If there is a deviation, the surgical catheter 300 can be adjusted immediately until the front-end of the surgical catheter 300 reaches around the surgical part A, and the excitation source 411 collaborated with the receiver 412 is used to obtain a surface image, a structure image or a dynamic structure image of the front-end tissue, thereby determining whether the surgical part A is reached. Therefore, the calibration of moving path of surgical catheter 300 can be performed precisely in accordance with the real-time tissue imaging, thus achieving an effect of correct positioning according an actual organizational condition. Once it is confirmed that the surgical catheter 300 reaches to the surgical part A and is positioned, the surgical end 510 of the surgical probe 500 will be stretchable freely in the hollow guide 301 of the surgical catheter 300 to directly reach the accurate surgical part A for performing various surgeries.

Based on the above embodiments, the advantages of the present invention are described as follows.
1. The image capturing system detects a tissue deformation or a displacement by comparing a tissue image captured by the image scanner before surgery with the actual tissue during surgery, thus confirming that the moving path of the surgical catheter is at the correct tissue site in accordance with the path plan in order to directly reach the optimal surgical site, and finally the navigator collaborated with the surgical probe is used for performing a precise positioning surgery.
2. The image capturing system captures a real-time surface image, a structure image or a dynamic structure image of the tissue, and the surgical catheter quickly transfers the image of the tissue to the surgical probe for the subsequent operation through the positioning of the image capturing system.
3. The working tube of the image capturing system showing a probe shape and flexibly collaborated with a hollow surgical catheter with two open ends and controllable curvature meets requirements of planning a path or adjusting a forward direction.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An image guided surgery apparatus, comprising:
a positioning frame;
a surgical catheter having a hollow guide with two open ends and controllable curvature, wherein the surgical catheter is disposed and positioned on the positioning frame; and
an image capturing system having a working tube and a main body which are connected, wherein the working tube shows a probe shape and is flexible for obtaining a real-time image, and the working tube is stretchable freely in the hollow guide of the surgical catheter.

2. The image guided surgery apparatus of claim 1, wherein the working tube of the image capture system is a real-time image capturing unit for capturing a surface image, a structure image or a dynamic structure image.

3. The image guided surgery apparatus of claim 1, wherein the working tube of the image capturing system uses an optical fiber technology to generate a surface image, a structure image or a dynamic structure image.

4. A probe image guided surgery system collaborated with a positioning frame, the probe image guided surgery system comprising:
an image scanner for obtaining at least one base image of an surgical part and generating a path plan;
a surgical catheter having a hollow guide with two open ends and controllable curvature, wherein the surgical catheter is disposed and positioned on the positioning frame, and one end of the surgical catheter reaches around the surgical part in accordance with the path plan;
an image capturing system having a working tube and a main body which are connected, wherein the working tube is stretchable freely in the hollow guide of the surgical catheter, and the working tube shows a probe shape and is flexible for capturing a real-time image in the front-end of the surgical catheter; and
a navigator connected to the image capturing system for displaying simultaneously or overlappingly the real-time image with the base image and the path planning obtained from the image scanner,

5. The probe image guided surgery system of claim 4, wherein the working tube of the image capturing system is a real-time image capturing unit for capturing a surface image, a structure image or a dynamic structure image.

6. The probe image guided surgery system of claim 5, wherein the image capturing unit comprises an excitation source and a receiver.

7. The probe image guided surgery system of claim 6, wherein the excitation source is used to emit visible light, invisible light, electromagnetic wave or supersonic wave.

8. The probe image guided surgery system of claim 6, wherein the receiver is used to receive a signal of visible light, invisible light, electromagnetic wave or supersonic wave through reflection or diffraction of a tissue at the surgical part.

9. The probe image guided surgery system of claim 6, wherein the main baby of the image capturing system generates and provides an excitation signal to the excitation source, and transforms a return signal from the receiver to a surface image, a structure image or a dynamic structure image of the tissue.
